(19) United States
(12) Patent Application Publication
(10) Pub. No.: US 2011/0224247 A1
(43) Pub. Date: Sep. 15, 2011

(54) AZAISOQUINOLINONE DERIVATIVES AS NK3 ANTAGONISTS

(75) Inventors: Nikolay Khanzhin, Humlebaek (DK); Klaus Bæk Simonsen, Odense M (DK); Søren Møller Nielsen, Hillerod (DK); Karsten Juhl, Greve (DK)

(73) Assignee: H. LUNDBECK A/S, Valby-Copenhagen (DK)

(21) Appl. No.: 13/045,743

(22) Filed: Mar. 11, 2011

Related U.S. Application Data
(60) Provisional application No. 61/313,441, filed on Mar. 12, 2010.

(51) Int. Cl. A61K 31/4375; C07D 471/04; A61P 25/18; A61P 25/24; A61P 25/28; A61P 25/16; A61P 25/22; A61P 25/08; A61P 11/14; A61P 11/06

(52) U.S. Cl. 514/300; 546/123

(57) ABSTRACT

The invention relates to compounds useful in therapy, in particular in the treatment of psychosis, to compositions comprising said compounds, and to methods of treating diseases comprising the administration of said compounds.

### CROSS-REFERENCE TO RELATED APPLICATIONS

The present application claims the benefit of priority under 35 U.S.C. §119(e) of U.S. Provisional Patent Application No. 61/313,441, filed Mar. 12, 2010, which is hereby incorporated by reference in its entirety.

### FIELD OF THE INVENTION

The present invention relates to compounds useful in therapy, in particular in the treatment of psychosis, to compositions comprising said compounds, and to methods of treating diseases comprising the administration of said compounds.

### BACKGROUND OF THE INVENTION

The currently approved antipsychotic drugs share the common feature of reducing dopamine signalling in the brain. This is achieved through either a dopamine D2 receptor antagonistic or partial agonistic effect. The first generation antipsychotics (also referred to as “typical”) are often associated with extra-pyramidal side effects for which reason the use of these agents has diminished. Second generation or “atypical” antipsychotics in addition to the D2 receptor affinity have affinity to the serotonin receptor 2A (5-HT_{2A}). Some atypical antipsychotics in addition have affinity for the 5-HT_{2C}, 5-HT₆, or 5-HT₇ receptors. Atypical antipsychotics give rise to fewer extra-pyramidal side effects, but are still hampered by weight gain and QT_{C} effects. Examples of atypicals are clozapine, olanzapine and risperidone.

More recently, neurokinin receptors have been suggested as targets for CNS diseases [Albert, Expert Opin. Ther. Patents, 14, 1421-1433, 2004]. Neurokinins (or tachykinins) are a family of neuropeptides which include substance P(SP), neurokinin A (NKA), and neurokinin B (NKB). The biological effects of these substances are primarily effected through binding to and activation of the three neurokinin receptors NK1, NK2, and NK3. Although some cross reactivity probably exists, SP has the highest affinity and is believed to be the endogenous ligand for NK1. Similarly, NKA is believed to be the endogenous ligand for NK2, and for NKB is believed to be the endogenous ligand for NK3.

NK3 is primarily expressed centrally in regions including cortical regions, such as frontal, parietal and cingulated cortex; nuclei of the amygdale, such as the basal, central and lateral nuclei; the hippocampus; and mesencephalon structures, such as ventral tegmental area, substantia nigra pars compacta, and dorsal raphe nuclei [Spooren et al, Nature Reviews, 4, 967-975, 2005]. The NK3 receptor is expressed on dopaminergic neurons, and Spooren et al has suggested that the antipsychotic effects of NK3 antagonists are mediated by an inhibition of the dopamine tone, particularly at the D2 receptor combined with a reduction of the serotonergic tone, particularly at the 5-HT_{2A} receptor.

Two structurally distinct NK3 antagonists, namely talnetant and osanetant, have been clinically tested for antipsychotic, and in particular antischizophrenic effects.

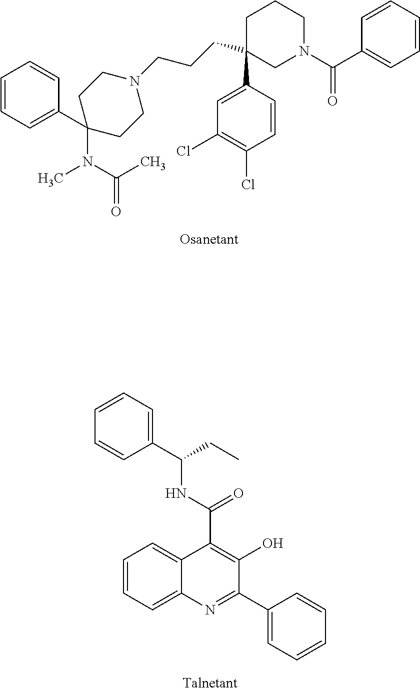

Osanetant proved superior to placebo in clinical trials, in particular on positive symptoms of psychosis, i.e. delusions, hallucinations and paranoia [Am. J. Psychiatry, 161, 2004, 975-984]. Similarly, talnetant has been shown in clinical trials to ameliorate the cognitive behaviour of schizophrenics [Curr. Opion. Invest. Drug, 6, 717-721, 2005]. Nevertheless, both compounds are hampered by poor pharmacokinetic and pharmacodynamic properties including poor solubility, poor bioavailability, relatively high clearance, and poor blood-brain barrier penetration [Nature reviews, 4, 967-975, 2005]. These results lend support to the notion that the NK3 receptor is a promising target for the treatment of e.g. psychosis, however emphasising the need for identifying compounds with adequate pharmacokinetic and pharmacodynamic properties.

WO95/32948 discloses a range of quinoline derivatives, including talnetant, as NK3 antagonists.

More recently, WO 2006/130080 discloses compounds having the core structure

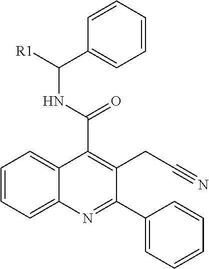

which compounds are said to be NK3 antagonists; and WO 2006/050991 and WO 2006/050992 disclose further quinolinecarboxamides derivatives, which derivatives are said to be NK3 antagonists.

Hence, there is a great desire for novel compounds, which are potent NK3 antagonists.

Also desired are novel compounds with improved properties relative to known compounds, which are NK3 antagonists, especially in relation to interactions with other medications.

### SUMMARY OF THE INVENTION

The present inventors have surprisingly found that certain azaisoquinolinone derivatives are potent NK3 antagonists which may as such be used in the treatment of e.g. psychosis. Accordingly, in one embodiment the invention relates to compounds of formula I

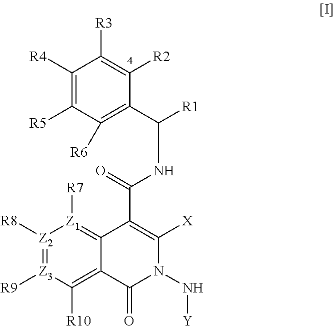

wherein R¹ is hydrogen or C₁₋₆alkyl;
X is H or C₁₋₆alkyl;
Y is C₁₋₆alkyl or haloC₁₋₆alkyl;
each of Z₁, Z₂ and Z₃ independently is C or N, provided that one of Z₁, Z₂ and Z₃ is N and provided that when Z₁ is N then Z₂ and Z₃ is C, when Z₂ is N then Z₁ and Z₂ is C, and when Z₃ is N then Z₁ and Z₂ is C;
each of R²-R⁶ and R¹⁰ independently is hydrogen or halogen;
R⁷ is hydrogen, halogen or when Z₁ is N, R⁷ is absent;
R⁸ is hydrogen, halogen or when Z₂ is N, R⁸ is absent;
R⁹ is hydrogen, halogen or when Z₃ is N, R⁹ is absent;
and pharmaceutically acceptable salts thereof.

In one embodiment, the invention relates to the use of a compound of formula I and pharmaceutically acceptable salts thereof in therapy.

In one embodiment, the invention relates to a pharmaceutical composition comprising compounds of formula I and pharmaceutically acceptable salts in combination with one or more pharmaceutically acceptable carrier or excipient.

In one embodiment, the invention relates to the use of a compound of formula I and pharmaceutically acceptable salts in the manufacture of medicaments.

In one embodiment, the invention relates to a compound of formula I and pharmaceutically acceptable salts for use in the treatment of diseases.

In one embodiment, the invention relates to a method a treatment, said method comprising the administration of a therapeutically effective amount of compound I and pharmaceutically acceptable salts to a patient in need thereof.

### DEFINITIONS

In the present context, “alkyl” is intended to indicate a straight, branched and/or cyclic saturated hydrocarbon. In particular “C₁₋₆alkyl” is intended to indicate such hydrocarbon having 1, 2, 3, 4, 5, or 6 carbon atoms. Examples of C₁₋₆alkyl include methyl, ethyl, propyl, butyl, pentyl, hexyl, cyclopropyl, cyclobutyl, cyclopentyl, cyclohexyl, 2-methyl-propyl, tert-butyl, and cyclopropylmethyl.

In the present context, “halogen” is intended to indicate members of the 7^{th} group of the periodic system, e.g. fluoro, chloro, bromo, and iodo.

In the present context, haloalkyl is intended to indicate an alkyl as defined above substituted with one or more halogens. In particular, haloC₁₋₆alkyl is intended to indicate a moiety wherein the alkyl part has 1, 2, 3, 4, 5 or 6 carbon atoms. One example of haloalkyl is trifluoromethyl.

In the present context, pharmaceutically acceptable salts include pharmaceutically acceptable acid addition salts. Acid addition salts include salts of inorganic acids as well as organic acids.

Examples of suitable inorganic acids include hydrochloric, hydrobromic, hydroiodic, phosphoric, sulfuric, sulfamic, nitric acids and the like.

Examples of suitable organic acids include formic, acetic, trichloroacetic, trifluoroacetic, propionic, benzoic, cinnamic, citric, fumaric, glycolic, itaconic, lactic, methanesulfonic, maleic, malic, malonic, mandelic, oxalic, picric, pyruvic, salicylic, succinic, methane sulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, theophylline acetic acids, as well as the 8-halotheophyllines, for example 8-bromotheophylline and the like. Further examples of pharmaceutically acceptable inorganic or organic acid addition salts include the pharmaceutically acceptable salts listed in J. Pharm. Sci. 1977, 66,2, which is incorporated herein by reference.

In the present context, the term “therapeutically effective amount” of a compound means an amount sufficient to cure, alleviate or partially arrest the clinical manifestations of a given disease and its complications in a therapeutic intervention comprising the administration of said compound. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician.

In the present context, the term “treatment” and “treating” means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications. Nonetheless, prophylactic (preventive) and therapeutic (curative) treatments are two separate aspects of the invention. The patient to be treated is preferably a mammal, in particular a human being.

### DETAILED DESCRIPTION OF THE INVENTION

In one embodiment, the invention provides compounds according to formula I

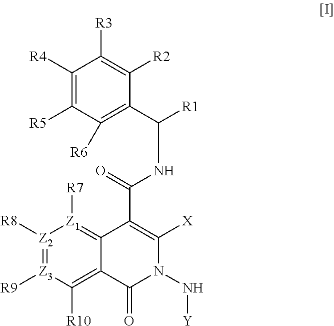

wherein R¹ is hydrogen or C₁₋₆alkyl;
X is H or C₁₋₆alkyl;
Y is C₁₋₆alkyl or haloC₁₋₆alkyl;
each of Z₁, Z₂ and Z₃ independently is C or N, provided that one of Z₁, Z₂ and Z₃ is N and provided that when Z₁ is N then Z₂ and Z₃ is C, when Z₂ is N then Z₁ and Z₂ is C, and when Z₃ is N then Z₁ and Z₂ is C;
each of R²-R⁶ and R¹⁰ independently is hydrogen or halogen;
R⁷ is hydrogen, halogen or when Z₁ is N, R⁷ is absent;
R⁸ is hydrogen, halogen or when Z₂ is N, R⁸ is absent;
R⁹ is hydrogen, halogen or when Z₃ is N, R⁹ is absent;
and pharmaceutically acceptable salts thereof (i.e. the compounds of the present invention).

In one embodiment, R¹ is C₁₋₆alkyl, in particular ethyl, cyclopropyl or cyclobutyl.

In one embodiment, X is C₁₋₆alkyl, in particular methyl.

In one embodiment, Y is C₁₋₆alkyl, in particular ethyl or propyl.

In one embodiment, Y is haloC₁₋₆alkyl, in particular fluoroC₁₋₆alkyl.

In one embodiment, Z₁ is N and Z₂ and Z₃ is C.

In one embodiment, Z₂ is N and Z₁ and Z₃ is C.

In one embodiment, Z₃ is N and Z₁ and Z₂ is C.

In one embodiment, at least one of R²-R⁶ and R¹⁰ is halogen.

In one embodiment, all of R²-R⁴ and R⁶ represent hydrogen.

In one embodiment, R⁵ is halogen, in particular fluorine or chlorine.

In one embodiment, R⁴ is halogen, in particular fluorine.

In one embodiment, R² is halogen, in particular fluorine.

In one embodiment, R¹⁰ is halogen, in particular fluorine.

In one embodiment, the invention provides compounds selected from the list
1a 2-Ethylamino-3-methyl-1-oxo-1,2-dihydro-[2,6]naphthyridine-4-carboxylic acid [(S)-cyclobutyl-(3-fluoro-phenyl)-methyl]-amide;
1b 3-Methyl-1-oxo-2-propylamino-1,2-dihydro-[2,6]naphthyridine-4-carboxylic acid [(S)-cyclopropyl-(4-fluoro-phenyl)-methyl]-amide;
1c 3-Methyl-1-oxo-2-propylamino-1,2-dihydro-[2,6]naphthyridine-4-carboxylic acid [(S)-(3-chloro-phenyl)-cyclopropyl-methyl]-amide;
1d 3-Methyl-1-oxo-2-propylamino-1,2-dihydro-[2,6]naphthyridine-4-carboxylic acid [cyclobutyl-(2-fluoro-phenyl)-methyl]-amide;
1e 3-Methyl-1-oxo-2-propylamino-1,2-dihydro-[2,6]naphthyridine-4-carboxylic acid [(S)-cyclobutyl-(3-fluoro-phenyl)-methyl]-amide;
1f 3-Methyl-1-oxo-2-propylamino-1,2-dihydro-[2,6]naphthyridine-4-carboxylic acid ((S)-cyclopropyl-phenyl-methyl)-amide;
1g 3-Methyl-1-oxo-2-propylamino-1,2-dihydro-[2,6]naphthyridine-4-carboxylic acid ((S)-1-phenyl-propyl)-amide;
2a 6-Ethylamino-7-methyl-5-oxo-5,6-dihydro-[1,6]naphthyridine-8-carboxylic acid [(S)-cyclobutyl-(3-fluoro-phenyl)-methyl]-amide; and
3a 2-Ethylamino-3-methyl-1-oxo-1,2-dihydro-[2,7]naphthyridine-4-carboxylic acid [(S)-cyclobutyl-(3-fluoro-phenyl)-methyl]-amide.

Further, the compounds of the invention may exist in unsolvated as well as in solvated forms in which the solvent molecules are selected from pharmaceutically acceptable solvents such as water, ethanol and the like. In general, such solvated forms are considered equivalent to the unsolvated forms for the purposes of this invention.

The compounds of the present invention may have one or more asymmetric centres and it is intended that any optical isomers (i.e. enantiomers or diastereomers), in the form of separated, pure or partially purified optical isomers and any mixtures thereof including racemic mixtures, i.e. a mixture of stereoisomers, are included within the scope of the invention. In particular, CH, indicated with an arrow, may be an asymmetrical centre giving rise to two optical isomers, an R form and an S form. In one embodiment, the compounds of the present invention have the S form.

In a particular embodiment, the compounds of the present invention have the following absolute configuration at CH, indicated with an arrow,

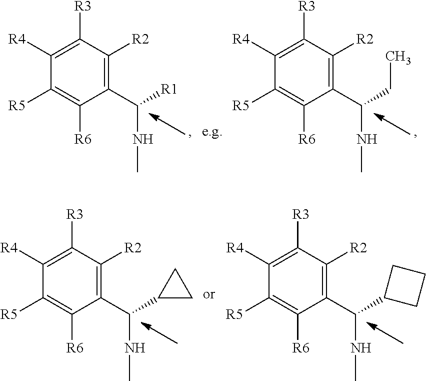

In this context is understood that when specifying the enantiomeric form, then the compound is in enantiomeric excess, e.g. essentially in a pure, mono-enantiomeric form. Accordingly, one embodiment of the invention relates to a compound of the invention having an enantiomeric excess of at least 60%, at least 70%, at least 80%, at least 85%, at least 90%, at least 96%, preferably at least 98%.

Racemic forms can be resolved into the optical antipodes by known methods, for example by separation of diastereomeric salts thereof with an optically active acid, and liberating the optically active amine compound by treatment with a base. Another method for resolving racemates into the optical antipodes is based upon chromatography of an optically active matrix. The compounds of the present invention may also be resolved by the formation of diastereomeric derivatives. Additional methods for the resolution of optical isomers, known to those skilled in the art, may be used. Such methods include those discussed by J. Jaques, A. Collet and S. Wilen in “Enantiomers, Racemates, and Resolutions”, John Wiley and Sons, New York (1981). Optically active compounds can also be prepared from optically active starting materials.

Furthermore, when a double bond or a fully or partially saturated ring system is present in the molecule geometric isomers may be formed. It is intended that any geometric isomers, as separated, pure or partially purified geometric isomers or mixtures thereof are included within the scope of the invention. Likewise, molecules having a bond with restricted rotation may form geometric isomers. These are also intended to be included within the scope of the present invention.

Furthermore, some of the compounds of the present invention may exist in different tautomeric forms and it is intended that any tautomeric forms that the compounds are able to form are included within the scope of the present invention.

NK3 receptor antagonists have been implicated in various diseases in addition to psychosis and schizophrenia discussed above. Langlois et al in J. Pharm. Exp. Ther., 299, 712-717, 2001, concludes that NK3 antagonists may be applicable in CNS diseases in general, and in anxiety and depression in particular. Yip et al in Br. J. Phar., 122, 715-722, 1997 further implicates NK3 antagonists in diverse brain functions, such as cortical processing, learning and memory, neuroendocrine and behavioral regulation. Additional studies have shown that NKB and NK3 receptors are involved in pain, and that NK3 antagonists have an antinociceptive and analgesic effect [Fioramonti, Neurogastroenterol. Motil., 15, 363-369, 2003]. Mazelin et al in Life Sci., 63, 293-304, 1998 show that NK3 antagonists have an effect in gut inflammation and concludes that such antagonists may be used in the treatment of irritable bowel syndrome (IBS). In addition, NK3 antagonists have in in vivo models been demonstrated to be useful in the treatment of airway related diseases, such as asthma, airway hyperresponsiveness, cough, and bronchorestriction [Daoui, Am. J. Respir. Crit. Care Med., 158, 42-48, 1998]. Maubach et al in Neurosci., 83, 1047-1062, 1998 show that NKB and the NK3 agonist senktide increase the frequency and duration of epileptiform discharges, and thus by inference that NK3 antagonists have a anticonvulsive potential. Finally, Kernel et al in J. Neurosci., 22, 1929-1936, 2002, suggests the use of NK3 antagonists in the treatment of Parkinson's Disease.

Accordingly, clinical, pre-clinical, in vivo and in vitro studies support that NK3 receptor antagonists are of relevance for the treatment or prevention of various disorders including psychosis, schizophrenia, depression, anxiety, cognitive impairment, obesity, Alzheimer's disease, Parkinson's disease, pain, convulsions, cough, asthma, airway hyperresponsiveness, microvascular hypersensitivity, bronchoconstriction, gut inflammation, inflammatory bowel syndrome, PTSD, dementia and agitation and delirium in the elderly.

Schizophrenia is classified into subgroups. The paranoid type is characterised by delusions and hallucinations and absence of thought disorder, disorganized behavior and affective flattening. The disorganized type, which is also named ‘hebephrenic schizophrenia’ in the ICD, in which thought disorder and flat affect are present together. The catatonic type, in which prominent psychomotor disturbances are evident, and symptoms may include catatonic stupor and waxy flexibility. The undifferentiated type in which psychotic symptoms are present but the criteria for paranoid, disorganized, or catatonic types have not been met. The symptoms of schizophrenia normally manifest themselves in three broad categories, i.e. positive, negative and cognitive symptoms. Positive symptoms are those, which represent an “excess” of normal experiences, such as hallucinations and delusions. Negative symptoms are those where the patient suffers from a lack of normal experiences, such as anhedonia and lack of social interaction. The cognitive symptoms relate to cognitive impairment in schizophrenics, such as lack of sustained attention and deficits in decision making. The current antipsychotics are fairly successful in treating the positive symptoms but fare less well for the negative and cognitive symptoms. Contrary to that, NK3 antagonists have been shown clinically to improve on both positive and negative symptoms in schizophrenics [Am. J. Psychiatry, 161, 975-984, 204], and according to the above discussion they are also expected to deliver an effect on the cognitive symptoms.

Cognitive impairment include a decline in cognitive functions or cognitive domains, e.g. working memory, attention and vigilance, verbal learning and memory, visual learning and memory, reasoning and problem solving e.g. executive function, speed of processing and/or social cognition. In particular, cognitive impairment may indicate deficits in attention, disorganized thinking, slow thinking, difficulty in understanding, poor concentration, impairment of problem solving, poor memory, difficulties in expressing thoughts and/or difficulties in integrating thoughts, feelings and behaviour, or difficulties in extinction of irrelevant thoughts.

In one embodiment, the present invention relates to the compounds of the present invention for use in therapy.

In one embodiment, the present invention relates to a method of treating a disease selected from psychosis; schizophrenia; schizophrenoform disorder; schizoaffective disorder; delusional disorder; brief psychotic disorder; shared psychotic disorder; psychotic disorder due to a general medical condition; substance or drug induced psychotic disorder (cocaine, alcohol, amphetamine etc); schizoid personality disorder; schizotypal personality disorder; psychosis or schizophrenia associated with major depression, bipolar disorder, Alzheimer's disease or Parkinson's disease; major depression; general anxiety disorder; bipolar disorder (maintenance treatment, recurrence prevention and stabilization); mania; hypomania; cognitive impairment; ADHD; obesity; appetite reduction; Alzheimer's disease; Parkinson's disease; pain; convulsions; cough; asthma; airway hyperresponsiveness; microvascular hypersensitivity; bronchoconstriction; chronic obstructive pulmonary disease; urinary incontinence; gut inflammation; inflammatory bowel syndrome; PTDS; dementia and agitation and delerium in the elderly the method comprising the administration of a therapeutically effective amount of a compound of the present invention to a patient in need thereof.

In one embodiment, the present invention relates to a method for the treatment of schizophrenia, the method comprising the administration of a therapeutically effective amount of a compound of the present invention to a patient in need thereof. In particular, said treatment includes the treatment of the positive, negative and/or cognitive symptoms of schizophrenia.

In one embodiment, the present invention relates to a method of treating cognitive impairment, the method comprising the administration of a therapeutically effective amount of a compound of the present invention to a patient in need thereof. In particular, said cognitive impairment is manifested as a decline in working memory, attention and vigilance, verbal learning and memory, visual learning and memory, reasoning and problem solving e.g. executive function, speed of processing and/or social cognition.

The antipsychotic effect of typical and atypical anti-psychotics, in particular D2 antagonists is exerted via an inhibition of the post-synaptic D2 receptors. Pre-synaptic D2 auto-receptors, however, are also affected by the administration of these compounds giving rise to an increase in the dopamine neuron firing rate, which, in fact, counteracts the antipsychotic effects. The increased firing rate continues until the effect of the pre-synaptic auto-receptors is blocked (the depolarization block), typically after approximately 3 weeks of chronic treatment with typical or atypical anti-psychotics. This model explains the up to 3 weeks delay of clinical effect normally seen when D2 antagonist treatment is initiated. NK3 antagonists seem to inhibit the increase in the dopamine neuron firing mediated by the pre-synaptic D2 auto-receptors brought about by D2 antagonists, wherefore the combined administration of NK3 antagonists (e.g. compounds of the present invention) and D2 antagonists is expected to give rise to a faster onset of the clinical effect. Moreover, D2 antagonists are known to increase prolactin levels, which may give rise to serious side effects, such as osteoporosis. It is known that NK3 agonists give rise to an increase in prolactin from which it may be deduced that a NK3 antagonist will lower an increased prolactin level, i.e bring about a normalisation of the prolactin level. A combined use of NK3 antagonists (e.g. compounds of the present invention) and D2 antagonists may thus address some of the safety issues associated with D2 antagonists administration. Similarly, NK3 antagonists (e.g. compounds of the present invention) may be administered together with antagonists/inverse agonists/negative modulators/partial agonists of one or more of the targets dopamine D2 receptor, dopamine D3 receptor, dopamine D4 receptor, phosphodiesterase PDE10, serotonin 5-HT_{1A} receptor, serotonin 5-HT_{2A} receptor, serotonin 5-HT₆ receptor, adrenergic alpha 2 receptor, cannabinoid type 1 receptor, histamine H3 receptor, cyclooxygenases, sodium channels or glycine transporter GlyT1; or with agonists/positive modulators/partial agonists of one or more of the targets serotonin 5-HT_{2C} receptor, KCNQ channels, NMDA receptor, AMPA receptor, nicotinic alpha-7 receptor, muscarinic M1 receptor, muscarinic M4 receptor, metabotropic glutamate receptor mGluR2, metabotropic glutamate receptor mGluR5, dopamine D1 receptor or dopamine D5 receptor.

Such combined administration of compounds of the present invention and other anti-psychotic compounds may be sequential or concomitant. Examples of D2 antagonists or partial agonists include haloperidol, chlorpromazine, sulpirid, risperidone, ziprasidon, olanzapine, quetiapin, clozapine and aripiprazole.

In one embodiment, the compound of the present invention is administered in an amount from about 0.001 mg/kg body weight to about 100 mg/kg body weight per day. In particular, daily dosages may be in the range of 0.01 mg/kg body weight to about 50 mg/kg body weight per day. The exact dosages will depend upon the frequency and mode of administration, the sex, the age the weight, and the general condition of the subject to be treated, the nature and the severity of the condition to be treated, any concomitant diseases to be treated, the desired effect of the treatment and other factors known to those skilled in the art.

A typical oral dosage for adults will be in the range of 1-1000 mg/day of a compound of the present invention, such as 1-500 mg/day, such as 1-100 mg/day or 1-50 mg/day.

In one embodiment, the present invention relates to the use of the compounds of the present invention in the manufacture of a medicament for the treatment of a disease selected from psychosis; schizophrenia; schizophrenoform disorder; schizoaffective disorder; delusional disorder; brief psychotic disorder; shared psychotic disorder; psychotic disorder due to a general medical condition; substance or drug induced psychotic disorder (cocaine, alcohol, amphetamine etc); schizoid personality disorder; schizotypal personality disorder; psychosis or schizophrenia associated with major depression, bipolar disorder, Alzheimer's disease or Parkinson's disease; major depression; general anxiety disorder; bipolar disorder (maintenance treatment, recurrence prevention and stabilization); mania; hypomania; cognitive impairment; ADHD; obesity; appetite reduction; Alzheimer's disease; Parkinson's disease; pain; convulsions; cough; asthma; airway hyperresponsiveness; microvascular hypersensitivity; bronchoconstriction; chronic obstructive pulmonary disease; urinary incontinence; gut inflammation; inflammatory bowel syndrome, PTSD; dementia and agitation and delirium in the elderly.

In one embodiment, the present invention relates to the use of a compound of the present invention in the manufacture of a medicament for the treatment of schizophrenia. In particular, said treatment includes the treatment of the positive, negative and/or cognitive symptoms of schizophrenia.

In one embodiment, the present invention relates to the use of a compound of the present invention in the manufacture of a medicament for the treatment of cognitive impairment. In particular, said cognitive impairment is manifested as a decline in working memory, attention and vigilance, verbal learning and memory, visual learning and memory, reasoning and problem solving e.g. executive function, speed of processing and/or social cognition.

In one embodiment, the present invention relates to a compound of the present invention for use in the treatment of a disease selected from psychosis; schizophrenia; schizophrenoform disorder; schizoaffective disorder; delusional disorder; brief psychotic disorder; shared psychotic disorder; psychotic disorder due to a general medical condition; substance or drug induced psychotic disorder (cocaine, alcohol, amphetamine etc); schizoid personality disorder; schizotypal personality disorder; psychosis or schizophrenia associated with major depression, bipolar disorder, Alzheimer's disease or Parkinson's disease; major depression; general anxiety disorder; bipolar disorder (maintenance treatment, recurrence prevention and stabilization); mania; hypomania; cognitive impairment; ADHD; obesity; appetite reduction; Alzheimer's disease; Parkinson's disease; pain; convulsions; cough; asthma; airway hyperresponsiveness; microvascular hypersensitivity; bronchoconstriction; chronic obstructive pulmonary disease; urinary incontinence; gut inflammation; inflammatory bowel syndrome; PTSD; dementia and agitation and delirium in the elderly.

In one embodiment, the present invention relates to a compound of the present invention for use in the treatment of schizophrenia. In particular, said treatment includes the treatment of the positive, negative and/or cognitive symptoms of schizophrenia.

In one embodiment, the present invention relates to a compound of the present invention for use in the treatment of cognitive impairment. In particular, said cognitive impairment is manifested as a decline in working memory, attention and vigilance, verbal learning and memory, visual learning and memory, reasoning and problem solving e.g. executive function, speed of processing and/or social cognition.

The compounds of the present invention may be administered alone as a pure compound or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19 Edition, Gennaro, Ed., Mack Publishing Co., Easton, Pa., 1995.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragees, pills, lozenges, powders and granules. Where appropriate, they can be prepared with coatings.

Liquid dosage forms for oral administration include solutions, emulsions, suspensions, syrups and elixirs.

Pharmaceutical compositions for parenteral administration include sterile aqueous and nonaqueous injectable solutions, dispersions, suspensions or emulsions as well as sterile powders to be reconstituted in sterile injectable solutions or dispersions prior to use.

Other suitable administration forms include suppositories, sprays, ointments, cremes, gels, inhalants, dermal patches, implants, etc.

Conveniently, the compounds of the invention are administered in a unit dosage form containing said compounds in an amount of about 0.1 to 500 mg, such as 1 mg, 5 mg, 10 mg, 50 mg 100 mg, 150 mg, 200 mg or 250 mg of a compound of the present invention.

For parenteral administration, solutions of the compound of the invention in sterile aqueous solution, aqueous propylene glycol, aqueous vitamin E or sesame or peanut oil may be employed. Such aqueous solutions should be suitably buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid and lower alkyl ethers of cellulose. Examples of liquid carriers are syrup, peanut oil, olive oil, phospho lipids, fatty acids, fatty acid amines, polyoxyethylene and water. The pharmaceutical compositions formed by combining the compound of the invention and the pharmaceutically acceptable carriers are then readily administered in a variety of dosage forms suitable for the disclosed routes of administration.

Formulations of the present invention suitable for oral administration may be presented as discrete units such as capsules or tablets, each containing a predetermined amount of the active ingredient, and which may include a suitable excipient. Furthermore, the orally available formulations may be in the form of a powder or granules, a solution or suspension in an aqueous or non-aqueous liquid, or an oil-in-water or water-in-oil liquid emulsion.

If a solid carrier is used for oral administration, the preparation may be tablet, e.g. placed in a hard gelatine capsule in powder or pellet form or in the form of a troche or lozenge. The amount of solid carrier may vary but will usually be from about 25 mg to about 1 g.

If a liquid carrier is used, the preparation may be in the form of a syrup, emulsion, soft gelatine capsule or sterile injectable liquid such as an aqueous or non-aqueous liquid suspension or solution.

Tablets may be prepared by mixing the active ingredient with ordinary adjuvants and/or diluents followed by the compression of the mixture in a conventional tabletting machine. Examples of adjuvants or diluents comprise: Corn starch, potato starch, talcum, magnesium stearate, gelatine, lactose, gums, and the like. Any other adjuvants or additives usually used for such purposes such as colourings, flavourings, preservatives etc. may be used provided that they are compatible with the active ingredients.

In one embodiment, the invention relates to a pharmaceutical composition comprising a compound of the present invention in combination with one or more pharmaceutically acceptable carrier or excipient.

In one embodiment, the invention relates to a pharmaceutical composition comprising a compound of the present invention together with a second anti-psychotic agent. In one embodiment, said second anti-psychotic agent is selected from antagonists/inverse agonists/negative modulators/partial agonists of the targets dopamine D2 receptor, dopamine D3 receptor, dopamine D4 receptor, phosphodiesterase PDE10, serotonin 5-HT_{1A} receptor, serotonin 5-HT_{2A} receptor, serotonin 5-HT₆ receptor, adrenergic alpha 2 receptor, cannabinoid type 1 receptor, histamine H3 receptor, cyclooxygenases, sodium channels or glycine transporter GlyT1; or from agonists/positive modulators/partial agonists of the targets serotonin 5-HT_{2C} receptor, KCNQ channels, NMDA receptor, AMPA receptor, nicotinic alpha-7 receptor, muscarinic M1 receptor, muscarinic M4 receptor, metabotropic glutamate receptor mGluR2, metabotropic glutamate receptor mGluR5, dopamine D1 receptor or dopamine D5 receptor. Particular examples of such anti-psychotics include haloperidol, chlorpromazine, sulpirid, risperidone, ziprasidon, olanzapine, quetiapine, clozapine and aripoprazole

In one embodiment, the invention relates to a pharmaceutical kit comprising a container containing a compound of the present invention and a separate container containing an anti-psychotic drug, such as typical anti-psychotics, atypical anti-psychotics, antagonists/inverse agonists/negative modulators/partial agonists of one or more of the targets dopamine D2 receptor, dopamine D3 receptor, dopamine D4 receptor, phosphodiesterase PDE10, serotonin 5-HT_{1A} receptor, serotonin 5-HT_{2A} receptor, serotonin 5-HT₆ receptor, adrenergic alpha 2 receptor, cannabinoid type 1 receptor, histamine H3 receptor, cyclooxygenases, sodium channels or glycine transporter GlyT1; or with agonists/positive modulators/partial agonists of one or more of the targets serotonin 5-HT_{2C} receptor, KCNQ channels, NMDA receptor, AMPA receptor, nicotinic alpha-7 receptor, muscarinic M1 receptor, muscarinic M4 receptor, metabotropic glutamate receptor mGluR2, metabotropic glutamate receptor mGluR5, dopamine D1 receptor or dopamine D5 receptor.

Particular examples of such anti-psychotics include haloperidol, chlorpromazine, sulpirid, risperidone, ziprasidon, olanzapine, quetiapine, clozapine and aripiprazole.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

The use of the terms “a” and “an” and “the” and similar referents in the context of describing the invention are to be construed to cover both the singular and the plural, unless otherwise indicated herein or clearly contradicted by context. For example, the phrase “the compound” is to be understood as referring to various “compounds” of the invention or a particular described aspect, unless otherwise indicated.

Unless otherwise indicated, all exact values provided herein are representative of corresponding approximate values (e.g., all exact exemplary values provided with respect to a particular factor or measurement can be considered to also provide a corresponding approximate measurement, modified by “about,” where appropriate).

The description herein of any aspect or aspect of the invention using terms such as “comprising”, “having,” “including,” or “containing” with reference to an element or elements is intended to provide support for a similar aspect or aspect of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

### Synthetic Routes

The compounds of the present invention of the general formula I, wherein R¹-R¹⁰, Z₁, Z₂, Z₃, X and Y are as defined above can be prepared by the methods outlined in the following reaction schemes and examples. In the described methods it is possible to make use of variants or modifications, which are themselves known to chemists skilled in the art or could be apparent to the person of ordinary skill in this art. Furthermore, other methods for preparing compounds of the invention will be readily apparent to the person skilled in the art in light of the following reaction schemes and examples. In the intermediate compounds of the general formulae II-XVII, R¹-R¹⁰, X and Y are as defined under formula I.

For compounds, which can exist as a mixture or equilibrium between two or more tautomers, only one tautomer is represented in the schemes, although it may not be the most stable tautomer. For compounds, which can exist in enantiomeric, stereoisomeric or geometric isomeric forms their geometric configuration is specified; otherwise the structure is a mixture of stereoisomers. Such compounds also include compounds of the present invention of the general formula I, which may exist as a mixture of atropisomers due to restricted rotation around carbon-carbon single bonds similar to atropisomerism in ortho, ortho′-disubstituted biaryl compounds also well-known to the person skilled in the art.

Starting materials of the general formulae V, VIII, XIV and XVII are obtained from commercial sources or they can be readily prepared by standard methods or their modifications described in the literature.

Compounds of formula I can be synthesized as shown in Scheme 1. Palladium catalysed aminocarbonylation of intermediate II using amines of general structure III and carbon monoxide gives intermediate IV, which on deprotection gives compounds of formula I. Amiens of formula III can be prepared as described below or are obtained from commercial sources.

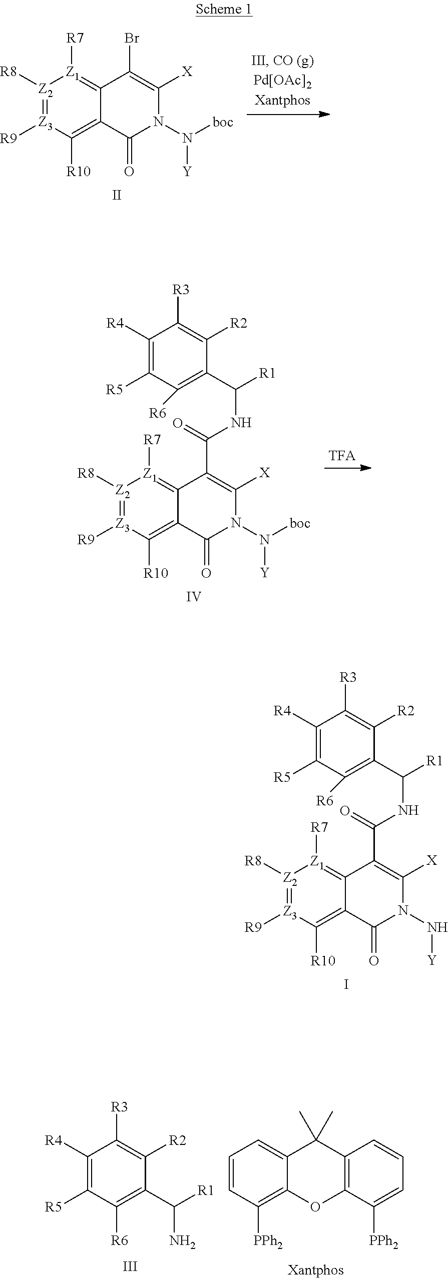

Intermediates of formula II can be synthesized as shown in Scheme 2. Palladium catalysed alkoxycarbonylation of intermediate V in the presence of an alcohol such as methanol and carbon monoxide gives intermediate VI. Hydrolysis of the ester moiety of VI gives intermediate VII. Double deprotonation of compound VII followed by quench with acyl donors such as Weinreb amides of formula VIII gives compounds of formula IX. Acid catalyzed ring-closure gives intermediates of formula X. Reactions of intermediates of formula X with tert-butyl carbazate give intermediates of formula XI, which on alkylation give intermediates of formula XII, followed by bromination with brominating reagents such as N-bromosuccinimide (NBS) to give intermediates of formula II.

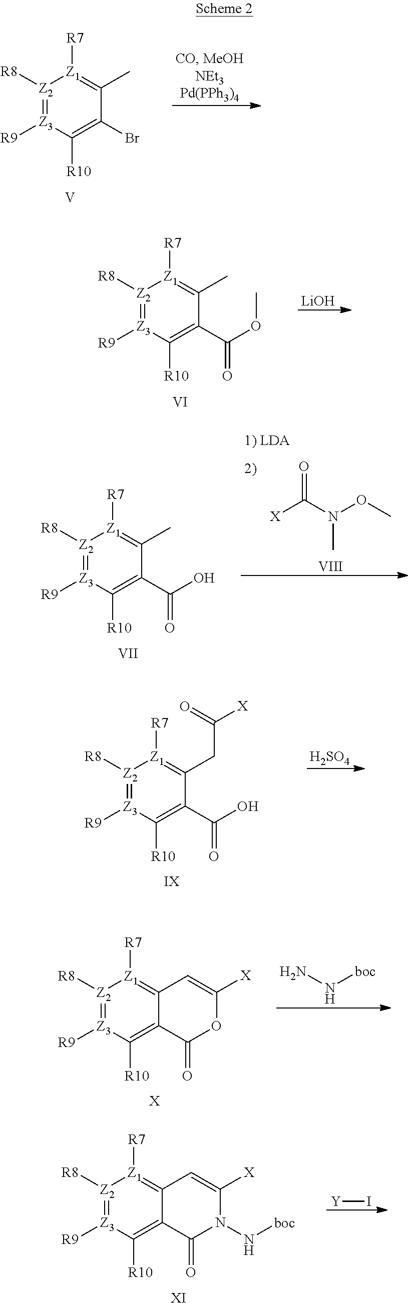

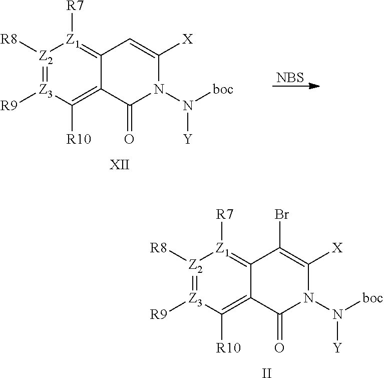

Compounds of formula I can also be synthesized as shown in Scheme 3. Hydrolysis of the ester moiety of intermediate XIII, followed by amide coupling using amines of formula III and coupling reagents such as 1-ethyl-3-(3-dimethylaminopropyl)carbodiimide hydrochloride/1-hydroxybenzotriazole gives intermediates IV, which on deprotection gives compounds of formula I.

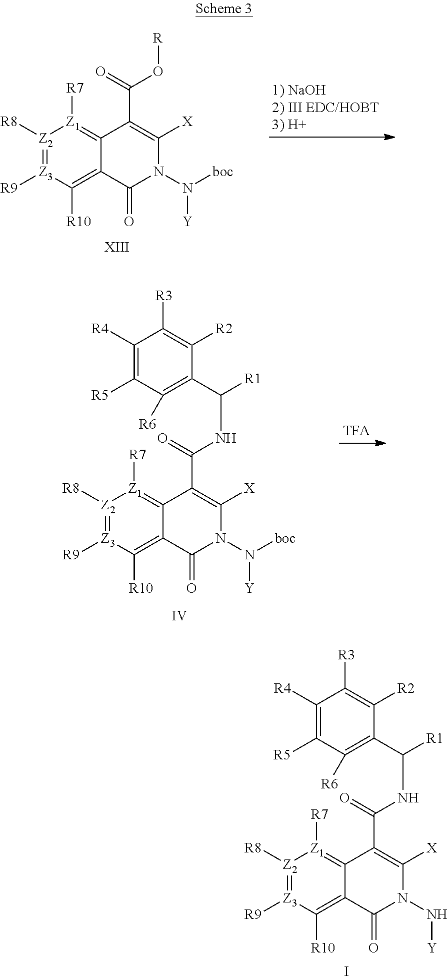

Intermediates of formula XIII can be synthesized as shown in Scheme 4. Amide couplings of intermediates of formula XIV, where LG is a leaving group such as fluorine, with protected hydrazines of formula XV using coupling reagents such as 1-ethyl-3-(3-dimethylaminopropyl)carbodiimide hydrochloride/1-hydroxybenzotriazole give intermediates of formula XVI. Deprotonation of the amide moiety of compounds of formula XVI and addition of alkynes of formula XVII gives a 1,4-addition to the alkyne followed by a ring closing nucleophilic aromatic substitution reaction to give intermediates of formula XIII. Protected hydrazines of formula XV can be synthesized by standard protection of the corresponding hydrazines, which are synthesized as described in Meyer K. G. Synlett 2004, 13, 2355.

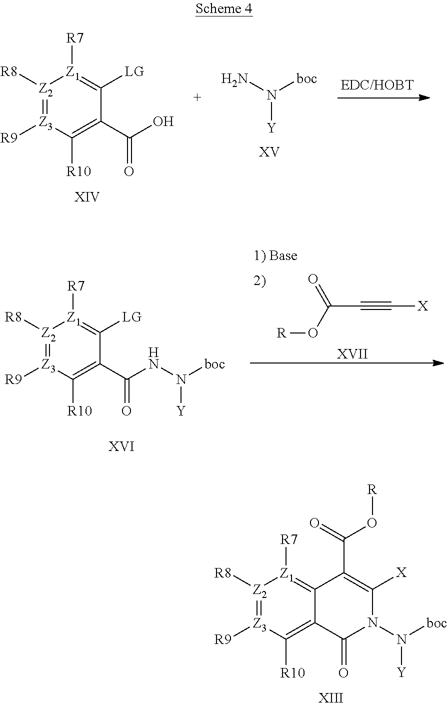

### EXAMPLES

Analytical LC-MS: data were obtained on a Sciex API 150EX analytical LC/MS system equipped with Applied Biosystems API150EX single qaudrupole mass spectrometer and atmospheric pressure photo ionisation (APPI) ion source, Shimadzu LC10ADvp LC pumps (3×), Shimadzu SPD-M20A photodiode array detector, SEDERE Sedex 85-low temperature Evaporative Light Scattering Detector (ELSD), Shimadzu CBM-20A system controller, Gilson 215 autosampler and Gilson 864 degasser controlled by Analyst Software. Column: 30×4.6 mm Waters Symmetry C18 column with 3.5 μm particle size; Injection Volume: 15 μL; Column temperature: 60° C.; Solventsystem: A=water/trifluoroacetic acid (100:0.05) and B=methanol with 0.035% trifluoroacetic acid;

### Gradient:

| | | |
|---|---|---|
| 0.01 min | 17% | B (v/v) |
| 0.27 min | 28% | B |
| 0.53 min | 39% | B |
| 0.80 min | 50% | B |
| 1.07 min | 59% | B |
| 1.34 min | 68% | B |
| 1.60 min | 78% | B |
| 1.87 min | 86% | B |
| 2.14 min | 93% | B |
| 2.38 min | 100% | B |
| 2.40 min | 17% | B |
| 2.80 min | 17% | B |

Total run time: 2.8 min

The retention times (t_{R}) are expressed in minutes based on UV-trace at 254 nm. Preparative LC-MS purification was performed on the same Sciex API 150EX system equipped with Gilson 333 and 334 pumps, Gilson GX 281 autosampler/fraction collector, Shimadzu LC10ADvp pump, Gilson UV/VIS 155 UV detector, Gilson 506C system interface, Gilson 864 degasser, Passive flowsplitters (approx. 1:1000). The MS and fraction collector were controlled by Masschrom software (Macintosh PC). The LC system was controlled by Trilution software version 2.0 (HP Compaq). The MS was controlled by Analyst (PC-Dell 390). For a small scale (<20 mg) purification fractions were collected in 4 ml vials using Sunfire Prep C18 5 μm, 10×100 mm, injection volume of 0-200 μL, flow rate of 15 ml/min and duration of 7.5 min, temperature +40° C. For a larger scale purification fractions were collected in 10 ml testtubes using Sunfire Prep C18 5 μm, 19×50 mm, injection volume of 0-200 μL, flow rate of 15 ml/min, temperature +40° C.

Solvents: A: Water containing 0.05% v/v TFA; B: Methanol containing 0.05% v/v TFA

| Time, min. | % B |
|---|---|
| 0.00 | 5.0 |
| 3.00 | 100.0 |
| 3.20 | 100.0 |
| 3.21 | 5.0 |

Microwave experiments were performed in sealed process vials or reactors using an Emrys Optimizer EXP from Personal Chemistry or a Milestone Microsynth instrument from Milestone.

### Preparation of Intermediates

### Synthesis of Chiral and Racemic Amines of the General Formula III

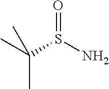

### (S)-(−)-2-Methyl-2-propanesulfinamide

The title chiral auxiliary was prepared according to a described procedure for the (R)-(+)-enantiomer by D. J. Weix and J. A. Ellman Organic Syntheses 2005, 82, 157.

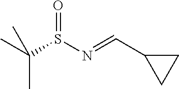

### (S)-2-Methyl-2-propanesulfinic acid 1-cyclopropyl-methylideneamide

The title compound was prepared according to a general procedure described by G. Liu, D. A. Cogan, T. D. Owens, T. P. Tang, and J. A. Ellman J. Org. Chem. 1999, 64, 1278: A mixture of cyclopropanecarboxaldehyde (35.0 g, 0.5 mol), (S)-(−)-2-methyl-2-propanesulfinamide (30 g, 0.25 mol) and anhydrous CuSO₄ (120 g, 0.75 mol) in CH₂Cl₂ (1500 mL) was stirred at room temperature overnight. The reaction mixture was filtered and evaporated to give the title compound (39 g, yield 95%), which was used in the next step without further purification.

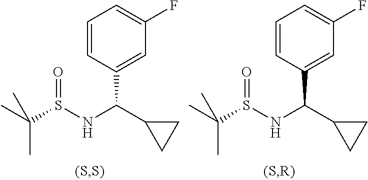

### (S)-2-Methyl-2-propanesulfinic acid [(S)-cyclopropyl-(3-fluoro-phenyl)-methyl]-amide and (S)-2-Methyl-2-propanesulfinic acid [(R)-cyclopropyl-(3-fluoro-phenyl)-methyl]-amide

The title compounds were obtained according to a general described procedure for 1,2-stereoselective addition of organometallic reagents to sulfinyl imines by D. A. Cogan, G. Liu, J. A. Ellman, Tetrahedron 1999, 55, 8883.

Procedure A: To an anhydrous lithium chloride (1.7 g, 40 mmol), THF (20 ml) was added under nitrogen followed by slow addition of i-PrMgCl (22 mL, 2 M in THF) and the obtained mixture was stirred at r.t. overnight. The obtained i-PrMgCl.LiCl solution was added dropwise to a stirred solution of 1-bromo-3-fluorobenzene (5.6 g, 33 mmol) in THF (25 ml) at 0° C. and stirring continued for 2 hours. The obtained Grignard reagent was added to a solution of (S)-2-methyl-2-propanesulfinic acid 1-cyclopropyl-methylideneamide (2.5 g, 14 mmol) in CH₂Cl₂ (60 mL) at −48° C. The mixture was stirred at −48° C. for 5 hours and then at room temperature overnight. The reaction mixture was quenched by addition of aq. sat. NH₄Cl (50 mL) and extracted with CH₂Cl₂ (3×100 mL). The combined organic solution was dried (Na₂SO₄) and evaporated to give a crude mixture, which was purified by column chromatography on silica gel (Ethyl acetate/petroleum ether=1/10). The obtained mixture of diastereoisomers was resolved by SFC to give the title (S,S)-isomer as the major product (1.5 g, yield 37.5%) and the title (S,R)-isomer (0.16 g, yield: 4.0%).

Procedure B: Alternatively, to a suspension of Mg (13.4 g, 0.55 mol) in 50 mL of anhydrous THF at 50° C. a solution of 1-bromo-3-fluorobenzene (89.0 g, 0.50 mol) was added dropwise. The mixture was stirred for 2 hours at 50° C. and then added dropwise to a solution of (S)-2-methyl-2-propanesulfinic acid 1-cyclopropyl-methylideneamide (78.0 g, 0.46 mol) in 100 mL of THF at 50-60° C. and stirred for 2 hours. It was quenched with aq. sat. NH₄Cl (100 ml), water (300 mL), filtered, and the solid and filtrate were extracted with hot ethyl acetate (600 mL) and evaporated in vacuo. The residue was crystallized from a mixture of ethyl acetate and petroleum ether (1:1, 200 mL) at −20° C. to give 80 g of the title (S,S)-isomer as a white powder, 66% yield, de 100% according to chiral HPLC. ¹H NMR (CDCl₃, 400 MHz, TMS=0 ppm): 7.34-7.28 (m, 1H), 7.16-7.12 (m, 2H), 7.00-6.96 (m, 1H), 3.68 (dd, J=8.8 Hz, 3.2 Hz, 1H), 3.52 (s, 1H), 1.42 (s, 9H), 1.15-1.08 (m, 1H), 0.84-0.75 (m, 1H), 0.69-0.61 (m, 1H), 0.55-0.46 (m, 1H), 0.28-0.21 (m, 1H).

### HCl

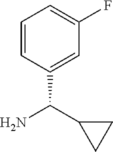

### (S)-(+)-C[C-Cyclopropyl-C-(3-fluoro-phenyl)]-methylamine hydrochloride

To a saturated solution of HCl in anhydrous dioxane (400 ml) (S)-2-methyl-2-propanesulfinic acid [(S)-cyclopropyl-(3-fluoro-phenyl)-methyl]-amide (80 g, 0.3 mol) was added at 0° C. The mixture was allowed to warm to r.t. After stirring for 1 hour, the reaction mixture was evaporated in vacuo. The residue was washed with anhydrous ether (2×100 ml) and dried in vacuo to give 56 g of the title compound as a white solid, yield 93%, ee >99.9% according to chiral HPLC. [α]_{D}²⁰=+52.69 (c=10 mg/mL, CH₃OH). ¹H NMR (CD₃OD, 400 MHz): 7.44-7.39 (m, 1H), 7.25-7.19 (m, 2H), 7.12-7.07 (m, 1H), 3.56 (d, J=10.0 Hz, 1H), 1.37-1.28 (m, 1H), 0.78-0.75 (m, 1H), 0.61-0.55 (m, 2H), 0.39-0.36 (m, 1H).

The following enantiomerically pure amine hydrochlorides were obtained analogously in three-step procedure starting from condensation of the corresponding aldehyde with chiral auxiliary, stereoselective Grignard addition where the mixture of diastereoisomers was resolved either by recrystallisation or by chromatography (SFC or column) and the major (S,S)-diastereoisomer was finally converted to a chiral amine with HCl.

| | | [α]²⁰D, | | |
|---|---|---|---|---|
| Structure | Chemical | (10 mg/ml) | ee (chiral | ¹H NMR |
| (HCl salt) | name | MeOH | HPLC) | (CD₃OD, 400 MHz) |
| 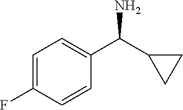 | C-[(S)-C- Cyclopropyl- C-(4-fluoro- phenyl)]- methylamine | +47.25 | 98.9 | 7.50-7.46(m, 2H), 7.19-7.14 (m, 2H), 3.56 (d, J = 10.0 Hz, 1H), 1.40-1.31 (m, 1H), 0.83-0.76 (m, 1H), 0.67-0.54 (m, 2H), 0.40-0.31 (m, 1H) |
| 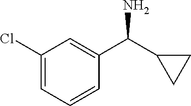 | C-[(S)-C-(3- Chloro- phenyl)-C- cyclopropyl]- methylamine | +54.25 | 96.9 | 7.55 (s, 1H),7.50- 7.42 (m, 3H), 3.61 (d, J = 10.0 Hz, 1H), 1.42-1.34 (m, 1H), 0.89-0.83 (m, 1H), 0.74-0.62 (m, 2H). |
| 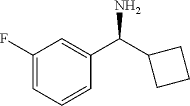 | C-[(S)-C- Cyclobutyl-C- (3-fluoro- phenyl)]- methylamine | +19.45 | 100 | 7.48-7.44 (m, 1H), 7.24-7.15 (m, 3H), 4.26 (d, J = 10.4 Hz, 1H), 2.87-2.84 (m, 1H), 2.25-2.24 (m, 1H), 2.05-1.76 (m, 5H). |

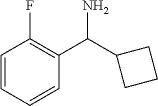

### C-Cyclobutyl-C-(2-fluoro-phenyl)-methylamine

One third of a solution of cyclobutyl bromide (5.0 g, 41.3 mmol) in anhydrous tetrahydrofuran (24 mL) was stirred with magnesium (1.11 g, 46.3 mmol) under reflux. The remaining solution was added dropwise over a period of 15 minutes, and the stirring at reflux continued for 30 min. To the obtained solution 2-fluorobenzonitrile (1.2 g) in THF (15 ml) was added dropwise at 0° C. The mixture was stirred for 5.5 h at 0° C. followed by addition of methanol (30 ml) and sodium borohydride (1.13 g). The reaction mixture was stirred for 16 hours at ambient temperature and concentrated. The residue was partitioned between chloroform (3×100 ml) and water, and the pH was adjusted to 1. The mixture was extracted with chloroform. The aqueous phase was adjusted to pH=10, and extracted with chloroform (3×100 ml). The combined organic layers were dried, evaporated and purified by column chromatography on silica gel (ethyl acetate/petroleum ether=1/1) to afford the title amine (0.45 g, yield: 7.9%) ¹H NMR (CD₃OD, 400 MHz) 7.49-7.38 (m, 2H), 7.30-7.15 (m, 2H), 4.50 (d, J=10.4 Hz, 1H), 3.00-2.90 (m, 1H), 2.29-2.21 (m, 1H), 2.09-1.71 (m, 5H).

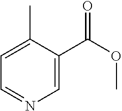

### 4-Methyl-nicotinic acid methyl ester

Triethylamine (45 g, 0.45 mol) was added into the mixture of compound 3-bromo-4-methyl-pyridine (25 g, 0.15 mol), Pd(OAc)₂ (3.37 g, 0.015 mol) and dppf (6.19 g, 0.015 mol) in DMF/MeOH (150 mL/150 mL) dropwise. Then the mixture was heated to 70˜80° C. under 50 psi of CO for 10 hours. The reaction mixture was cooled to room temperature and filtered. The filtrate was concentrated in vacuo and the residue was purified by chromatography on silica gel (petroleum ether/ethyl acetate=10:1) to afford 15 g of 4-methyl-nicotinic acid methyl ester as a yellow oil in 66% yield.

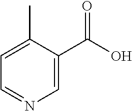

### 4-Methyl-nicotinic acid

4-Methyl-nicotinic acid methyl ester (15 g, 0.1 mol) was added into the mixture of aqueous NaOH (2 M, 55 mL) and dioxane (100 mL), then the reactant was heated to 50˜60° C. for 1 hour. The solvent was removed in vacuo. The residue was diluted with water (20 mL) and acidified by 1N HCl to PH≈6, the solid formed was filtered and dried in vacuo to give 8.0 g of 4-methyl-nicotinic acid as a white solid in 58.4% yield.

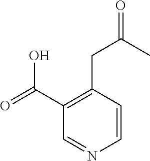

### 4-(2-Oxo-propyl)-nicotinic acid

To 4-methyl-nicotinic acid (1.37 g, 10 mmol) in THF (75 mL) at −78° C., lithium diisopropyl amide (0.020 mol, prepared from a 2M solution of n-butyl lithium in hexane, 0.02 mol and diisopropyl amine, 0.02 mol) was added dropwise, and the solution was stirred at −78° C. for 0.5 hour, the mixture was warmed up to −20° C. for 0.5 hours, and then cooled to −78° C., a solution of N-methoxy-N-methyl acetamie (1.03 g, 10 mmol) in THF (50 mL) was added at −78° C. After the addition was complete, the mixture was stirred at −78° C. for 0.5 hours, then allowed to warm to room temperature and stirred for another 2 hours, and then water (10 mL) was added. The reaction mixture was concentrated in vacuo and cooled to form a solid, which was used for the next step directly.

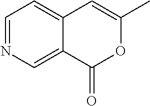

### 3-Methyl-pyrano[3,4-c]pyridin-1-one

4-(2-Oxo-propyl)-nicotinic acid (0.8 g, 5 mmol) in 30% H₂SO₄ (10 mL) was stirred at room temperature overnight, then the mixture was carefully basified with saturated aqueous NaHCO₃, extracted with dichloro methane (30 mL×2). The combined organic fractions were dried over Na₂SO₄, concertrated in vacuo, and purified by flash chromatography on silica gel(petroleum ether/ethyl acetate=3/1) to give 0.4 g of 3-methyl-pyrano[3,4-c]pyridin-1-one as a white solid in 50% yield.

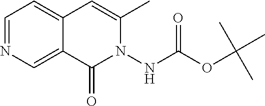

### (3-Methyl-1-oxo-1H-[2,7]naphthyridin-2-yl)-carbamic acid tert-butyl ester

3-Methyl-pyrano[3,4-c]pyridin-1-one (0.4 g, 2.5 mmol) and tert-butyl carbazate (0.97 g, 7.5 mmol) in ethanol (5 mL) were heated at reflux for 13 hours. The solvent was removed in vacuo and the residue was purified by preperative TLC (petroleum ether/ethyl acetate=1/1) to give 0.1 g of (3-methyl-1-oxo-1H-[2,7]naphthyridin-2-yl)-carbamic acid tert-butyl ester as a grey solid in 14.5% yield.

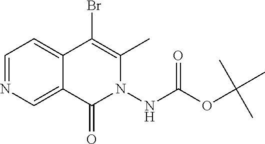

### (4-Bromo-3-methyl-1-oxo-1H-[2,7]naphthyridin-2-yl)-carbamic acid tert-butyl ester

N-Bromosuccinimide (1.07 g, 5.99 mmol) was added to a solution of (3-methyl-1-oxo-1H-[2,7]naphthyridin-2-yl)-carbamic acid tert-butyl ester (1.5 g, 5.4 mmol) in dimethylformamide (10 mL) at room temperature. The reaction mixture was stirred at room temperature for 2 hours. The reaction mixture was poured into water (20 mL), and extracted with dichloromethane (30 mL×2), the combined organic layers were dried over Na₂SO₄, concertrated in vacuo, and the residu was purified by preparative HPLC to give 1.1 g of (4-bromo-3-methyl-1-oxo-1H-[2,7]naphthyridin-2-yl)-carbamic acid tert-butyl ester as a white solid in 57.3% yield. ¹H NMR (400 MHz, CDCl₃): δ9.44 (s, 1H), 8.75 (d, J=5.6 Hz, 1H), 7.62 (d, J=6.0 Hz, 1H), 7.40 (br, 1H), 2.61 (s, 3H), 1.45 (s, 9H).

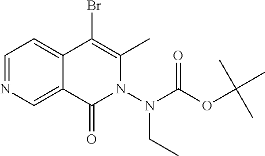

### (4-Bromo-3-methyl-1-oxo-1H-[2,7]naphthyridin-2-yl)-ethyl-carbamic acid tert-butyl ester

Iodoethane (114 uL, 1.43 mmol) was added to (4-bromo-3-methyl-1-oxo-1H-2,7-naphthyridin-2-yl)-carbamic acid tert-butyl ester (507 mg, 1.43 mmol) and potassium carbonate (396 mg, 2.86 mmol) in N-methylpyrrolidinone (4.0 mL, 41 mmol). The reaction mixture was stirred at 50° C. for 3 days. The reaction mixture was concentrated in vacuo. 50 mL water was added. The mixture was extracted with ethyl acetate (3×50 mL). The organic phases were pooled and washed with brine, dried over MgSO₄ and concentrated in vacuo. The crude product was purified by flash chromatography on silica gel (eluent: heptane/ethyl acetate) to obtain the product.

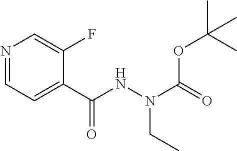

### N-Ethyl-N′-(3-fluoro-pyridine-4-carbonyl)-hydrazinecarboxylic acid tert-butyl ester

N-(3-Dimethylaminopropyl)-N′-ethylcarbodiimide hydrochloride (2.05 g, 10.7 mmol) and 4-dimethylaminopyridine (1.30 g, 10.7 mmol) were added to 3-fluoroisonicotinic acid (1.37 g, 9.71 mmol) in methylene chloride (35 mL). The reaction mixture was stirred for 10 minutes. N-ethyl-hydrazinecarboxylic acid tert-butyl ester (1.71 g, 10.7 mmol) was added. The reaction mixture was stirred at room temperature for 3 days. The reaction mixture was concentrated to ca. 10 mL and was purified by flash chromatography to give N-ethyl-N′-(3-fluoro-pyridine-4-carbonyl)-hydrazinecarboxylic acid tert-butyl ester (yield: 0.92 g, 33%).

¹H NMR (600 MHz, CDCl₃) δ 8.64 (d, J=1.3 Hz, 1H), 8.61 (d, J=4.6 Hz, 1H), 8.31 (s, J=84.3 Hz, 1H), 7.92 (s, 1H), 3.66 (q, J=7.2 Hz, 2H), 1.47 (s, 9H), 1.20 (t, J=7.2 Hz, 3H).

The following compounds were synthesized analogously:

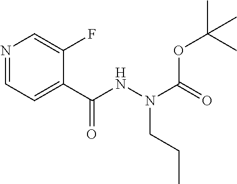

### N′-(3-Fluoro-pyridine-4-carbonyl)-N-propyl-hydrazinecarboxylic acid tert-butyl ester

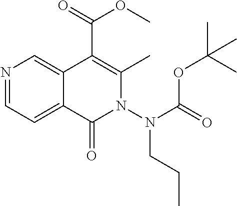

### 2-(tert-Butoxycarbonyl-propyl-amino)-3-methyl-1-oxo-1,2-dihydro-2,6-naphthyridine-4-carboxylic acid ethyl ester

0.6 M Sodium bis(trimethylsilyl)amide in toluene (1.5 mL) was added to N′-(3-fluoro-pyridine-4-carbonyl)-N-propyl-hydrazinecarboxylic acid tert-butyl ester (243 mg, 0.817 mmol) in N,N-dimethylformamide (6.3 mL) under an atmosphere of Argon, and the mixture was stirred for 1 hour at room temperature. Ethyl 2-butynoate (191 uL, 1.63 mmol) was added dropwise to the reaction mixture, and after stirring for 1 hour the mixture was heated under microwave irradiation (for 6 hours at 100° C.). The reaction mixture was poured into water (100 mL) and extracted with an ethyl acetate:diethyl ether mixture (1:1, 200 mL). The organic phase was washed with diluted brine (water:sat. NaCl(aq)=1:1, 4×100 mL), dried over MgSO₄ and concentrated in vacuo. The product was purified by flash chromatography to give the title compound (yield: 67 mg, 21%).

¹H NMR (600 MHz, CDCl₃) δ 9.34 (d, J=7.6 Hz, 1H), 8.69 (dd, J=10.9, 5.5 Hz, 1H), 8.35 (dd, J=7.0, 2.4 Hz, 1H), 4.57-4.46 (m, 2H), 3.91-3.74 (m, 1H), 3.49-3.36 (m, 1H), 2.53 (d, J=7.2 Hz, 3H), 1.71-1.61 (m, 1H), 1.61-1.51 (m, 6H), 1.46 (dt, J=16.1, 7.2 Hz, 3H), 1.35 (s, 5H), 0.93 (dt, J=11.4, 7.4 Hz, 3H).

¹³C NMR (151 MHz, CDCl₃) δ 158.55, 153.73, 145.93, 145.65, 142.49, 141.87, 129.52, 122.29, 83.49, 82.81, 62.48, 53.12, 51.69, 28.18, 27.94, 21.49, 20.98, 17.27, 14.23, 11.35.

The following compound was synthesized analogously:

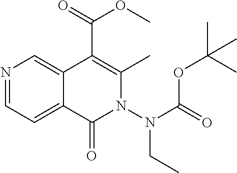

### 6-(tert-Butoxycarbonyl-ethyl-amino)-7-methyl-5-oxo-5,6-dihydro-1,6-naphthyridine-8-carboxylic acid methyl ester

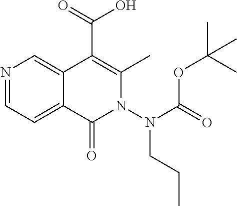

### 2-(tert-Butoxycarbonyl-propyl-amino)-3-methyl-1-oxo-1,2-dihydro-2,6-naphthyridine-4-carboxylic acid

2-(tert-Butoxycarbonyl-propyl-amino)-3-methyl-1-oxo-1,2-dihydro-2,6-naphthyridine-4-carboxylic acid ethyl ester (0.26 g, 0.67 mmol) was dissolved in methanol (4 mL). 2 M of sodium hydroxide in water (1.0 mL) was added and the reaction mixture was stirred overnight at r.t. 1M HCl(aq) is added to the reaction mixture until pH=3-4. The mixture was extracted with ethyl acetate. The organic phase was dried with MgSO₄ and solvents were removed by evaporation to give crude 2-(tert-butoxycarbonyl-propyl-amino)-3-methyl-1-oxo-1,2-dihydro-2,6-naphthyridine-4-carboxylic acid, which was used in the next step without further purification.

The following compounds were synthesized analogously:

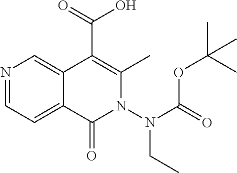

### 2-(tert-Butoxycarbonyl-ethyl-amino)-3-methyl-1-oxo-1,2-dihydro-[2,6]naphthyridine-4-carboxylic acid

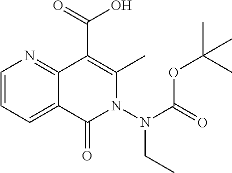

### 6-(tert-Butoxycarbonyl-ethyl-amino)-7-methyl-5-oxo-5,6-dihydro-[1,6]naphthyridine-8-carboxylic acid

### Preparation of Compounds of the Invention

### Example 1

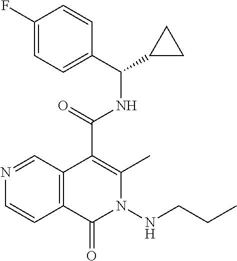

### 1b 3-Methyl-1-oxo-2-propylamino-1,2-dihydro-[2,6]naphthyridine-4-carboxylic acid [(S)-cyclopropyl-(4-fluoro-phenyl)-methyl]-amide

2-(tert-Butoxycarbonyl-propyl-amino)-3-methyl-1-oxo-1,2-dihydro-2,6-naphthyridine-4-carboxylic acid (10 mg, 0.03 mmol) and C—[(S)—C-cyclopropyl-C-(4-fluoro-phenyl)]-methylamine (6.8 mg, 0.042 mmol) were dissolved in N,N-dimethylformamide (0.3 mL, 4 mmol). 1-Hydroxybenzotriazole (5.6 mg, 0.042 mmol) was added. N-(3-Dimethylaminopropyl)-N′-ethylcarbodiimide hydrochloride (8.0 mg, 0.042 mmol) was added. Triethylamine (12 uL, 0.083 mmol) was added. The reaction mixture was stirred overnight at room temperature. Dichloromethane (150 μL) and trifluoro acetic acid (150 μL) were added. The reaction mixture was stirred at room temperature for 2 hours. 150 μL Trifluoro acetic acid was added. The reaction mixture was stirred at 50° C. for 1 hour. The reaction mixture was concentrated in vacuo and the product was purified by preparative HPLC.

LC-MS (m/z) 409.8 (MH+); t_{R}=1.67

The following compounds were synthesized analogously:

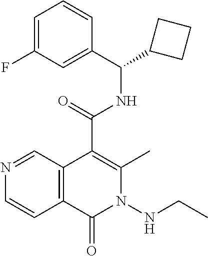

### 1a 2-Ethylamino-3-methyl-1-oxo-1,2-dihydro-[2,6]naphthyridine-4-carboxylic acid [(S)-cyclobutyl-(3-fluoro-phenyl)-methyl]-amide

LC-MS (m/z) 409.3 (MH+); t_{R}=1.61

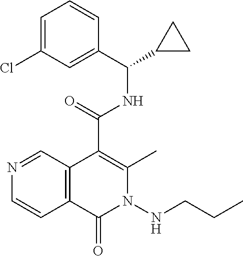

### 1c 3-Methyl-1-oxo-2-propylamino-1,2-dihydro-[2,6]naphthyridine-4-carboxylic acid [(S)-(3-chloro-phenyl)-cyclopropyl-methyl]-amide

LC-MS (m/z) 425.6 (MH+); t_{R}=1.77

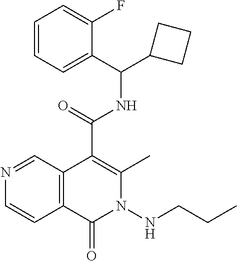

### 1d 3-Methyl-1-oxo-2-propylamino-1,2-dihydro-[2,6]naphthyridine-4-carboxylic acid [cyclobutyl-(2-fluoro-phenyl)-methyl]-amide

LC-MS (m/z) 423.5 (MH+); t_{R}=1.77

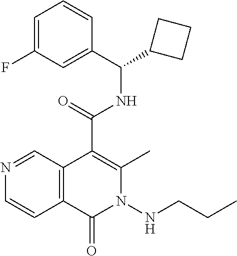

### 1e 3-Methyl-1-oxo-2-propylamino-1,2-dihydro-[2,6]naphthyridine-4-carboxylic acid [(S)-cyclobutyl-(3-fluoro-phenyl)-methyl]-amide

LC-MS (m/z) 423.4 (MH+); t_{R}=1.79

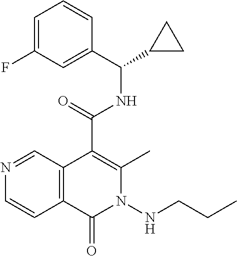

### 1f 3-Methyl-1-oxo-2-propylamino-1,2-dihydro-[2,6]naphthyridine-4-carboxylic acid ((S)-cyclopropyl-phenyl-methyl)-amide

LC-MS (m/z) 391.9 (MH+); t_{R}=1.63

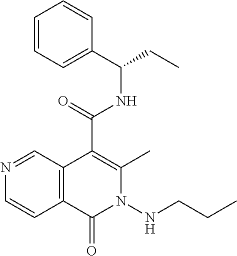

### 1g 3-Methyl-1-oxo-2-propylamino-1,2-dihydro-[2,6]naphthyridine-4-carboxylic acid ((S)-1-phenyl-propyl)-amide

LC-MS (m/z) 379.8 (MH+); t_{R}=1.60

### Example 2

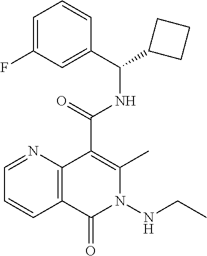

### 2a 6-Ethylamino-7-methyl-5-oxo-5,6-dihydro-[1,6]naphthyridine-8-carboxylic acid [(S)-cyclobutyl-(3-fluoro-phenyl)-methyl]-amide

LC-MS (m/z) 409.5 (MH+); t_{R}=1.73

### Example 3

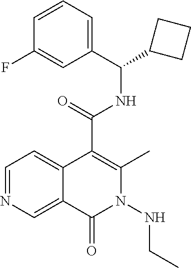

### 3a 2-Ethylamino-3-methyl-1-oxo-1,2-dihydro-2,7-naphthyridine-4-carboxylic acid [(S)-cyclobutyl-(3-fluoro-phenyl)-methyl]-amide

4-Bromo-3-methyl-1-oxo-1H-2,7-naphthyridin-2-yl)-ethyl-carbamic acid tert-butyl ester (65 mg, 0.17 mmol) and C—[(S)—C-Cyclobutyl-C-(3-fluoro-phenyl)]-methylamine (45.7 mg, 0.255 mmol) were added to palladium(II) acetate (3.82 mg, 0.0170 mmol), 4,5-bis-diphenylphosphanyl-9,9-dimethyl-9H-xanthene (9.84 mg, 0.0170 mmol), and sodium carbonate (54.1 mg, 0.510 mmol) in Toluene (1 mL, 10 mmol). The reaction mixture was stirred under an atmosphere of Carbon Monoxide (2 bar) at 120° C. overnight. The reaction mixture was cooled to room temperature. Ethanol was added (20 mL). The mixture was filtered. The residue was suspended in water (200 mL) and ethyl acetate (200 mL). The mixture was acidified carefully with conc. HCl(aq). The mixture was filtered. The organic phase of the filtrate was washed with brine, dried over MgSO₄ and concentrated in vacuo and purified by flash chromatography to give (4-{[(S)-cyclobutyl-(3-fluoro-phenyl)-methyl]-carbamoyl}-3-methyl-1-oxo-1H-2,7-naphthyridin-2-yl)-ethyl-carbamic acid tertbutyl ester.

It was dissolved in dichloromethane (1 mL). Trifluoro acetic acid (1 mL) was added. The reaction mixture was stirred at room temperature for 1 hour. The reaction mixture was concentrated in vacuo and purified by preperative TLC to give the title compound (yield 8.4 mg, 12%).

LC-MS (m/z) 409.4 (MH+); t_{R}=1.56

¹H NMR (600 MHz, DMSO) δ 9.38 (s, 1H), 9.01 (d, J=8.6 Hz, 1H), 8.68 (s, 1H), 7.41 (td, J=7.9, 6.2 Hz, 1H), 7.21 (d, J=7.7 Hz, 2H), 7.20-7.17 (m, 1H), 7.10 (td, J=8.3, 2.0 Hz, 1H), 6.34 (s, 1H), 5.03 (dd, J=9.8, 8.9 Hz, 1H), 3.07-2.80 (m, 2H), 2.68-2.55 (m, 1H), 2.37 (m, 2H), 2.06 (dt, J=10.9, 7.7 Hz, 1H), 1.93 (dd, J=11.5, 7.4 Hz, 1H), 1.81 (m, Hz, 2H), 1.74 (m, 1H), 1.31-1.19 (m, 1H), 1.17 (t, J=7.1 Hz, 3H).

### Example 4

### NK3 Receptor Binding Assay

Membranepreparation: BHK cells stably expressing the human NK3 receptor were seeded in harvesting plates in Dulbeccos MEM containing GlutaMax (862 mg/l), 1 mM sodium pyruvate, 10% fetal calf serum, 1% Pen/Strep, 1 mg/ml G418 and were grown at 34° C. in a humidified atmosphere containing 10% CO₂. To increase receptor expression, 10 μM trichotatin A was added to the media 24 hours before harvest of the cells at a confluency of app 90%. Prior to the harvesting, the cells were washed twice with PBS without Mg²⁺ and Ca²⁺ and subsequently scrapped of in 10 ml PBS pr harvesting plate. The cells suspension were centrifuged at 1500×G in three minutes before resuspension in 15 mM Tris-HCl pH 7,5 buffer containing 2 mM MgCl₂; 0,3 mM EDTA and 1 mM EGTA (buffer A). The cell suspention was homogenised and subsequently centrifuged at 40000×G in 30 minutes. The membrane-pellet was resuspended in buffer A containing 250 mM sucrose, alliquoted and stored at −80° C.

Affinity assay description: The assay is performed as a filter-based competition-binding in a 50 mM Tris pH 7.4 assay buffer containing 120 mM NaCl and 3 mM MnCl₂. App 0.05 nM ³H-AE93103 was mixed with test compounds before addition of 0.15 μg of a homogenised NK3 membrane preparation in a total volume of 300 μl. The assay plate is incubated for 90 min at RT before content of the wells is transferred using a cell-harvester to GF/C filter plates, which has been pretreated with 0.1% PEI. The filter is washed 3 times with 1 ml an ice-cold 50 mM Tris buffer, pH 7,4. The filter is dried and added scintillation liquid before the plate is counted in a topcounter 5 minutes pr well.

The total binding, which comprised less than 10% of added radioligand, was defined using assay buffer whereas the non-specific binding was defined in the presence of 1 μM SR142801. The non-specific binding constituted ˜10% of the total binding. Data points are expressed in percent of the specific binding of ³H-AE93103 and the 1050 values (concentration causing 50% inhibition of ³H-AE93103 specific binding) are determined by non-linear regression analysis using a sigmoidal variable slope curve fitting. The dissociation constant (Ki) were calculated from the Cheng Prusoff equation (Ki=IC₅₀/(1+(L/K_{d})), where the concentration of free radioligand L is approximated to the concentration of added ³H-AE93103 in the assay (˜0.05 nM) and Kd equals the affinity of the ³H-AE93103 for the NK3 receptor. The Kd of ³H-AE93103 was determined to be 0.072 nM from four independent saturation assays each performed with duplicate determinations. Bmax was ˜15 pmol/mg.

The compounds of the present invention generally have Kᵢ values of 1000 nM or less, such as 500 nM or less, such as 200 nM or less.

### Results

Kᵢ values of all azaisoquinolinone derivatives of the present invention are listed in table 1.

TABLE 1

| Example | Affinity (Kᵢ/nM) |
|---|---|
| 1a | 58 |
| 31b | 720 |
| 1c | 540 |
| 1d | 160 |
| 1e | 33 |
| 1f | 330 |
| 1g | 430 |
| 2a | 91 |
| 3a | 110 |

1. A compound according to formula I 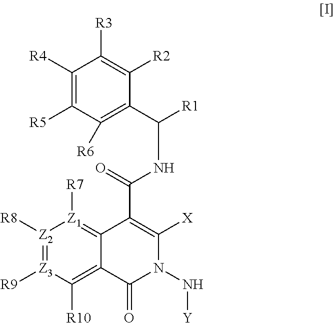
wherein R¹ is hydrogen or C₁₋₆alkyl;
X is H or C₁₋₆alkyl;
Y is C₁₋₆alkyl or haloC₁₋₆alkyl;
each of Z₁, Z₂ and Z₃ is independently C or N, provided that one of Z₁, Z₂ or Z₃ is N, and provided that when Z₁ is N then Z₂ and Z₃ is C, when Z₂ is N then Z₁ and Z₂ is C, and when Z₃ is N then Z₁ and Z₂ is C;
each of R²-R⁶ and R¹⁰ is independently hydrogen or halogen;
R⁷ is hydrogen or halogen, or when Z₁ is N, R⁷ is absent;
R⁸ is hydrogen or halogen, or when Z₂ is N, R⁸ is absent;
R⁹ is hydrogen or halogen, or when Z₃ is N, R⁹ is absent;
and pharmaceutically acceptable salts thereof.

2. The compound according to claim 1, wherein R¹ is C₁₋₆alkyl.

3. The compound according to claim 2, wherein R¹ is ethyl, cyclopropyl or cyclobutyl.

4. The compound according to claim 1, wherein X is C₁₋₆alkyl.

5. The compound according to claim 4, wherein X is methyl.

6. The compound according to claim 1, wherein Y is C₁₋₆alkyl.

7. The compound according to claim 6, wherein Y is ethyl or propyl.

8. The compound according to claim 1, wherein Y is haloC₁₋₆alkyl.

9. The compound according to claim 8, wherein Y is fluoroC₁₋₆alkyl.

10. The compound according to claim 1, wherein Z₁ is N, and both Z₂ and Z₃ are C.

11. The compound according to claim 1, wherein Z₂ is N, and both Z₁ and Z₃ are C.

12. The compound according to claim 1, wherein Z₃ is N, and both Z₁ and Z₂ are C.

13. The compound according to claim 1, wherein at least one of R²-R⁶ and R¹⁰ is halogen.

14. The compound according to claim 1, wherein all of R²-R⁴ and R⁶ are hydrogen.

15. The compound according to claim 1, wherein R⁵ is halogen.

16. The compound according to claim 15, wherein R⁵ is fluorine and chlorine.

17. The compound according to claim 1, wherein R⁴ is halogen.

18. The compound according to claim 17, wherein R⁴ is fluorine.

19. The compound according to claim 1, wherein R² is halogen.

20. The compound according to claim 19, wherein R² is fluorine.

21. The compound according to claim 1, wherein R¹⁰ is halogen.

22. The compound according to claim 21, wherein R¹⁰ is fluorine.

23. The compound according to claim 1 selected from the group consisting of:
2-Ethylamino-3-methyl-1-oxo-1,2-dihydro-[2,6]naphthyridine-4-carboxylic acid [(S)-cyclobutyl-(3-fluoro-phenyl)-methyl]-amide;
3-Methyl-1-oxo-2-propylamino-1,2-dihydro-[2,6]naphthyridine-4-carboxylic acid [(S)-cyclopropyl-(4-fluoro-phenyl)-methyl]-amide;
3-Methyl-1-oxo-2-propylamino-1,2-dihydro-[2,6]naphthyridine-4-carboxylic acid [(S)-(3-chloro-phenyl)-cyclopropyl-methyl]-amide;
3-Methyl-1-oxo-2-propylamino-1,2-dihydro-[2,6]naphthyridine-4-carboxylic acid [cyclobutyl-(2-fluoro-phenyl)-methyl]-amide;
3-Methyl-1-oxo-2-propylamino-1,2-dihydro-[2,6]naphthyridine-4-carboxylic acid [(S)-cyclobutyl-(3-fluoro-phenyl)-methyl]-amide;
3-Methyl-1-oxo-2-propylamino-1,2-dihydro-[2,6]naphthyridine-4-carboxylic acid ((S)-cyclopropyl-phenyl-methyl)-amide;
3-Methyl-1-oxo-2-propylamino-1,2-dihydro-[2,6]naphthyridine-4-carboxylic acid ((S)-1-phenyl-propyl)-amide;
6-Ethylamino-7-methyl-5-oxo-5,6-dihydro-[1,6]naphthyridine-8-carboxylic acid [(S)-cyclobutyl-(3-fluoro-phenyl)-methyl]-amide; and
2-Ethylamino-3-methyl-1-oxo-1,2-dihydro-[2,7]naphthyridine-4-carboxylic acid [(S)-cyclobutyl-(3-fluoro-phenyl)-methyl]-amide;
or pharmaceutically acceptable salts thereof.

24. A pharmaceutical composition comprising a compound according to claim 1 in combination with one or more pharmaceutically acceptable carriers or excipients.

25. A pharmaceutical composition comprising a compound according to claim 23 in combination with one or more pharmaceutically acceptable carriers or excipients.

26. A method for the treatment of a disease selected from the group consisting of: psychosis; schizophrenia; schizophrenoform disorder; schizoaffective disorder; delusional disorder; brief psychotic disorder; shared psychotic disorder; psychotic disorder due to a general medical condition; substance or drug induced psychotic disorder; schizoid personality disorder; schizotypal personality disorder; psychosis or schizophrenia associated with major depression, bipolar disorder, Alzheimer's disease or Parkinson's disease; major depression; general anxiety disorder; bipolar disorder (maintenance treatment, recurrence prevention and stabilization); mania; hypomania; cognitive impairment; ADHD; obesity; appetite reduction; Alzheimer's disease; Parkinson's disease; pain; convulsions; cough; asthma; airway hyperresponsiveness; microvascular hypersensitivity; bronchoconstriction; chronic obstructive pulmonary disease; urinary incontinence; gut inflammation; inflammatory bowel syndrome; PTSD; and dementia and agitation and delirium in the elderly; wherein the method comprises administration of a therapeutically effective amount of a compound according to claim 1 to a patient in need thereof.

27. The method according to claim 26, wherein the disease is schizophrenia.

28. The method according to claim 27, wherein positive, negative and/or cognitive symptoms are treated.

29. A method for the treatment of a disease selected from the group consisting of: psychosis; schizophrenia; schizophrenoform disorder; schizoaffective disorder; delusional disorder; brief psychotic disorder; shared psychotic disorder; psychotic disorder due to a general medical condition; substance or drug induced psychotic disorder; schizoid personality disorder, schizotypal personality disorder; psychosis or schizophrenia associated with major depression, bipolar disorder, Alzheimer's disease or Parkinson's disease; major depression; general anxiety disorder; bipolar disorder (maintenance treatment, recurrence prevention and stabilization); mania; hypomania; cognitive impairment; ADHD; obesity; appetite reduction; Alzheimer's disease; Parkinson's disease; pain; convulsions; cough; asthma; airway hyperresponsiveness; microvascular hypersensitivity; bronchoconstriction; chronic obstructive pulmonary disease; urinary incontinence; gut inflammation; inflammatory bowel syndrome; PTSD; and dementia and agitation and delirium in the elderly, wherein the method comprises administration of a therapeutically effective amount of a compound according to claim 23 to a patient in need thereof.

30. The method according to claim 29, wherein the disease is schizophrenia.

31. The method according to claim 30, wherein positive, negative and/or cognitive symptoms are treated.
